(19) United States
(12) Patent Application Publication
(10) Pub. No.: US 2010/0035867 A1
(43) Pub. Date: Feb. 11, 2010

(54) Inhibitors of Cyclic Nucleotide Synthesis and Their Use for Therapy of Various Diseases

(76) Inventors: Richard L. Guerrant, Charlottesville, VA (US); Alexander Y. Kots, Houston, TX (US); Ferid Murad, Houston, TX (US); Byung-Kwon Choi, Katy, TX (US)

Correspondence Address: WILLIAMS, MORGAN & AMERSON, 10333 RICHMOND, SUITE 1100, HOUSTON, TX, 77042, US

(21) Appl. No.: 12/307,150

(22) PCT Filed: Jul. 6, 2007
(86) PCT No.: PCT/US07/72929

§ 371 Date: Oct. 12, 2009

(30) Foreign Application Priority Data
Jul. 11, 2006 (US) 60806942

(51) Int. Cl. A61K 31/5415; A61K 31/519

(52) U.S. Cl. 514/225.8; 514/257

(57) ABSTRACT

We disclose a method of inhibiting activity of adenylyl cyclase or guanylyl cyclase in a mammal by administering to the mammal an amount of a composition effective to inhibit the activity, wherein the composition contains at least one compound selected from the group consisting of structural formulae (Ia) and (Ib) and salts thereof, wherein R1 is —H or has the structure —C(═O)R8; R2 is ═O or has the structure —OC(═O)R9; and R3, R4, R5, R6, and R7 are each independently selected from the group consisting of —H, —NO₂, formula (I), -halogen, —OC(═O)R9, —OR9, —OH, —R8OH, —CH₃, —OC(═O)CH₂Ph, formulae (II), (III), (IV), —OPh, —CF₃, —R8, —C(═O)OR9, -Ph, —R8Ph, formulae (V), (VI), (VII), (VIII), (IX), (X), (XI), (XII), (XIII), (XIV), (XV), (XVI), (XVII), (XVIII), (XIX), (XX), and (XXI), wherein each R8 is independently a linear or branched hydrocarbon group having from 1 to 4 carbon atoms and each R9 is independently a hydrocarbon group having from 1 to 2 carbon atoms. Administering the composition can be used to treat a disease in a mammal mediated by activity of adenylyl cyclase or guanylyl cyclase and effected by a toxin produced by a pathogenic organism or to reduce cyclic AMP or cyclic GMP levels in a mammal in need of reduction thereof. The composition can also be administered to mammalian cells in vitro. The above methods of inhibiting activity of adenylyl cyclase or guanylyl cyclase and treating diseases via such inhibition can be effective without prolonged treatment, have reversible effects, have low or no toxicity, are highly potent, are unlikely to have side effects, do not act on purinergic receptors, or can negate pathogenic toxins independently of whether the pathogenic organism survives.

### BACKGROUND OF THE INVENTION

The development of this invention was funded in part by The Robert A. Welch Foundation grant no. AU-1437.

The present invention relates generally to the field of medicinal chemistry. More particularly, it concerns inhibitors of cyclic nucleotide synthesis and their use in treating various diseases.

Cyclic nucleotides are synthesized by the enzymes adenylyl cyclase and guanylyl cyclase. Cyclic nucleotides are important messengers which regulate the cellular functions. The synthesis of cyclic nucleotides is activated by various hormones, drugs, and other intracellular and extracellular agents. This results in an increase in the intracellular amount of cyclic nucleotides. Thus, inhibitors of adenylyl cyclase or guanylyl cyclase can decrease the amount of intracellular cyclic nucleotides.

In various diseases, such as cholera, the synthesis of cyclic nucleotides is activated, thus promoting the activity of various targets, including protein kinases, which can activate other molecules, such as the chloride anion transporter cystic fibrosis transmembrane conductance regulator (CFTR). FIG. 1 provides an overview of the cyclic nucleotide synthesis process and indicates points where various agents can activate cyclic nucleotide synthesis.

Diarrhea is a common medical condition which has considerable contribution to morbidity, loss of work productivity, and consumption of medical resources. Over a billion people suffer at least one episode of acute diarrhea each year. Acute infectious diarrhea is the most common cause of mortality in developing countries accounting for 5 to 8 million deaths of children each year.

Acute diarrhea is also a leading cause of death in younger cattle, piglets, and other domestic animals causing significant economic loss to farmers and ranchers. Neonatal colibacillary diarrhea in newborn farm animals is the most common enteric disease.

More than 90% of cases of acute diarrhea in man and lower animals are caused by various infectious agents. Among these infectious agents are certain strains of bacteria which produce specific toxins. These toxins play a major role in pathogenesis of diarrhea.

Escherichia coli is a gram-negative bacterial pathogen responsible for deaths of hundreds of thousands children per year in developing countries.

Pathogenic mechanisms in E. coli-induced diarrhea may involve secretion of a heat stable toxin STa and a heat-labile toxin LT. STa and LT specifically influence ion transport in intestinal mucosa.

STa binds to a receptor protein on enterocytes. This protein is called guanylyl cyclase type C. Binding of STa to guanylyl cyclase C stimulates synthesis of cyclic GMP by the enzyme. Cyclic GMP in turn activates a C1⁻-transporter in the intestinal brush border which results in excessive accumulation of electrolytes and water in the intestinal lumen. This is the mechanism of diarrhea induced by STa.

LT and the toxin from Vibrio cholerae (cholera toxin) bind to a cell surface receptor. Then, these toxins penetrate inside the cell and induce indirect activity of adenylyl cyclase which synthesizes cyclic AMP. Cyclic AMP in turn activates a C1⁻-transporter and this activation results in diarrhea as described in a preceding paragraph.

Similar mechanisms of regulation of intestinal secretion are involved in the effects of other hormones and mediators inducing diarrhea such as activators of guanylyl cyclase and adenylyl cyclase produced endogenously in the body.

It is known that in various cells and tissues, stimulation of cyclic nucleotide accumulation can result in altered regulatory pathways which can be linked to transport of ions through the membrane of these or other cells and hence induce a number of pathological conditions.

Various bacterial toxins can induce an increase in the amount of cyclic nucleotides in the cells and tissues. These toxins include adenylyl cyclase toxins of Bordetella pertussis and Bordetella parapertussis and other similar toxins of Bordetella sp., Exo Y toxin of Pseudomonas aeruginosa and other similar toxins of representatives of the bacterial family Pseudomonadaceae, adenylyl cyclase of Yersinia pestis and other similar proteins of the Yersinia sp., and adenylyl cyclase (edema factor) which is the component of the edema toxin of Bacillus anthracis.

A number of attempts to treat diseases by administering compounds which interact with adenylyl cyclase or guanylyl cyclase are known. Parkinson et al. used 2-chloroadenosine to suppress the effects of ST toxin from E. coli (Parkinson, S. J.; Alekseev, A. E.; Gomez, L. A.; Wagner, F.; Terzic, A.; Waldman, S. A., Interruption of Escherichia coli heat-stable enterotoxin-induced guanylyl cyclase signaling and associated chloride current in human intestinal cells by 2-chloroadenosine. J Biol Chem 1997, 272, (2), 754-8). Cells or tissues were treated with 2-chloroadenosine which was then converted inside the cells to 2-chloroATP and then 2-chloroATP suppressed the activity of guanylyl cyclase type C.

However, this approach had a number of disadvantages. First, the agent used by Parkinson et al. required prolonged treatment of cells and tissues (around 24 hours) to biotransform the substance into an actual inhibitor to have an inhibitory activity. Second, the inhibitory effects of 2-chloroadenosine were irreversible and that compound could not be washed or otherwise removed from cells and tissues. Third, 2-chloroadenosine is a toxic compound and can inhibit other enzymes and proteins which use cellular ATP for their function. Fourth, the potency of 2-chloroadenosine (IC50) is only about 50 μM. Fifth, 2-chloroadenosine is partially water-soluble and thus can penetrate into various cells and tissues (carried there with the blood flow) which are not desired to be the targets of its action, suggesting side effects may occur. Sixth, 2-chloroadenosine readily acts on a number of purinergic receptors; thus, it can affect various endogenous processes associated with purinergic receptors. These receptors are extremely important and critical for various functional parameters of the organism.

A second attempt is related to a very dangerous infectious disease, anthrax. A common method for therapy of anthrax involves treatment of infected animals or human patients with antibiotics. However, huge amount of the toxins produced by Bacillus anthracis in the host still can kill the host even after all bacteria can be eliminated with such treatment. This accounts for the very high lethality of the disseminated disease.

Therefore, it would be desirable to have methods of treating diseases involving activity of adenylyl cyclase or guanylyl cyclase which may be effective with one or more of shorter treatment, more reversible effects, lower toxicity, higher potency, lower likelihood of side effects, less activation of purinergic receptors, or more negation of pathogenic toxins independently of whether the pathogenic organism survives. It would be desirable to have methods which may be effective with two or more thereof

### SUMMARY OF THE INVENTION

In one embodiment, the present invention relates to a method of inhibiting activity of adenylyl cyclase or guanylyl cyclase in a mammal by administering to the mammal an amount of a composition effective to inhibit the activity, wherein the composition contains at least one compound selected from the group consisting of structural formulae Ia and Ib and salts thereof:

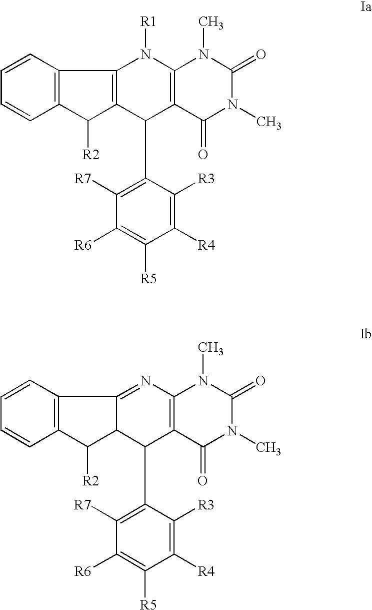

wherein R1 is —H or has the structure —C(═O)R8;

R2 is ═O or has the structure —OC(═O)R9; and

R3, R4, R5, R6, and R7 are each independently selected from the group consisting of —H, —NO₂, -halogen, —OC(═O)R9, —OR9, —OH, —R8OH, —CH₃, —OC(═O)CH₂Ph,

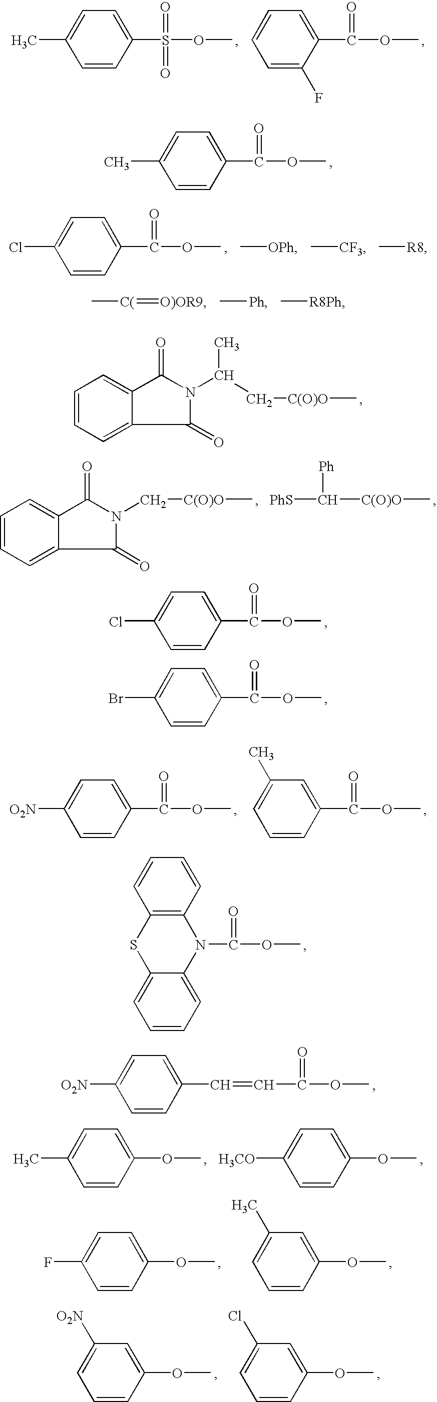

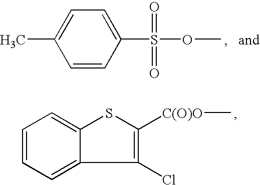

wherein each R8 is independently a linear or branched hydrocarbon group having from 1 to 4 carbon atoms and each R9 is independently a hydrocarbon group having from 1 to 2 carbon atoms.

In one embodiment, the present invention relates to a method of treating a disease in a mammal mediated by activity of adenylyl cyclase or guanylyl cyclase and effected by a toxin produced by a pathogenic organism by administering to the mammal an amount of a composition effective to treat the disease, wherein the composition contains at least one compound selected from the group consisting of structural formulae Ia and Ib and salts thereof, as described above.

In one embodiment, the present invention relates to a method of reducing cyclic AMP or cyclic GMP levels in a mammal in need of reduction thereof by administering to the mammal an amount of a composition effective to reduce the cyclic AMP or cyclic GMP levels, wherein the composition contains at least one compound selected from the group consisting of structural formulae Ia and Ib and salts thereof, as described above.

In one embodiment, the present invention relates to a method of inhibiting activity of adenylyl cyclase or guanylyl cyclase in mammalian cells in vitro by administering to the mammalian cells an amount of a composition effective to inhibit the activity, wherein the composition contains at least one compound selected from the group consisting of structural formulae Ia and Ib and salts thereof, as described above.

The above methods of inhibiting activity of adenylyl cyclase or guanylyl cyclase and treating diseases via such inhibition may be effective with one or more of shorter treatment, more reversible effects, lower toxicity, higher potency, lower likelihood of side effects, less activation of purinergic receptors, or more negation of pathogenic toxins independently of whether the pathogenic organism survives.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an overview of the cyclic nucleotide synthesis process and indicates points where various disease toxins can activate cyclic nucleotide synthesis. “In” and “out” refer to inside and outside a cell. A stimulatory ligand (Ls), by binding to a stimulatory GTP-binding protein-coupled receptor (Rs), induces activation of the GTP-binding stimulatory protein (Gs), which subsequently causes activation of adenylyl cyclase (AC) and results in increased synthesis of cyclic adenosine monophosphate (cAMP); cholera toxin causes activation of Gs also leading to increase in cAMP synthesis and forskolin is capable of direct activation of AC. Certain bacterial toxins including adenylyl cyclase toxin from Bordetella pertussis (BAC) and edema factor from Bacillus anthracis (EF) possesss an intrinsic adenylyl cyclase activities and also increase cAMP after penetrating inside an animal cell. A number of agents including stable toxin a from E. coli (STa) and hormones guanylin and uroguanylin stimulate the activity of guanylyl cyclase type C (GC-C) to increase production of another cyclic nucleotide, cyclic guanosine monophosphate (cGMP); similar increase in cGMP can be induced by activation of guanylyl cyclase type A (GC-A) with atrial natriuretic peptide (ANP) and brain natriuretic peptide (BNP) or by activation of guanylyl cyclase type B (GC-B) with C-type natriuretic peptide (CNP) as well as by activation of soluble guanylyl cyclase (sGC) by nitric oxide (NO). Increased intracellular levels of cAMP result in activation of cAMP-dependent protein kinase A (PKA) and increased intracellular levels of cGMP result in activation of cGMP-dependent protein kinase G (PKG). Upon stimulation, these protein kinases phosphorylate cystic fibrosis transmembrane conductance regulator (CFTR) thus enhancing the flux of cloride anions (Cl⁻) through the membrane to the extracellular medium.

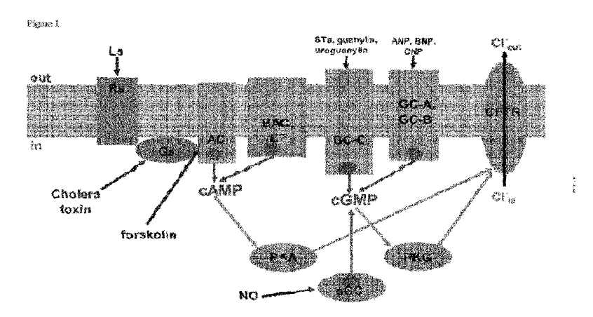

FIG. 2 shows inhibition of STa-induced activation of GC-C in T84 cells by compounds IIa, IIb, IIIa, IIIb, IVb, Vb, VI, and VII as identified in Example 1 and according to the experiment reported in Example 2.

FIG. 3 shows inhibition of STa-induced activation of GC-C in T84 cells by 50 μM compound IIb (BPIPP) at various concentrations of STa starting from 1 nM and gradually increasing up to 5 μM according to the experiment reported in Example 2.

FIG. 4 shows inhibition of guanylin-induced and STa-induced activation of GC-C in T84 cells by various concentrations of compound IIb (BPIPP) at 0.5 μM guanylin and 0.1 μM STa according to the experiments reported in Example 2 and Example 3.

FIG. 5 shows the effects of various concentrations of compound IIb (BPIPP) on activation of membrane guanylyl cyclase by 1 μM atrial natriuretic peptide (ANP) and 0.5 μM C-type natriuretic peptide in human neuroblastoma BE-2 cells according to the experiments reported in Example 4.

FIG. 6 shows inhibition of cyclic GMP accumulation by 50 μM compound IIb (BPIPP) in rat fetal lung fibroblast RFL-6 cells stimulated with 10 μM nitric oxide donor benzotrifuroxan, 1 μM ANP, and 1 μM CNP according to the experiments reported in Example 4.

FIG. 7 shows inhibition of cyclic AMP accumulation in T84 cells treated with cholera toxin (CT), adenylyl cyclase toxin of Bordetella pertussis (BAC), and edema toxin of Bacillus anthracis (ET) by exposure to 50 μM compound IIb (BPIPP) present during infection phase, incubation phase, and both infection and incubation phase according to the experiments reported in Examples 5, 14, and 15.

FIG. 8 shows inhibition of forskolin, isoproterenol, or cholera toxin-induced activation of adenylyl cyclase in rat fetal lung fibroblast RFL-6 cells by compound IIb according to the experiment reported in Example 6.

FIG. 9 shows lack of the effect of 50 μM compound Ia (BPIPP) on extrusion of cyclic GMP from T84 cells stimulated with 100 nM STa according to experiment reported in Example 9.

FIG. 10 shows inhibition of forskolin-induced chloride transport in T84 cells by compound IIb according to the experiment reported in Example 10.

FIG. 11 shows the effects of 50 μM compound IIb (BPIPP) on stimulation of chloride efflux from T84 cells induced by 100 μM isoproterenol (Iso), 10 μg/ml cholera toxin (CT; CT-inf, compound IIb was present during infection; CT-inc, compound IIb was present during incubation; CT-both, compound IIb was present during both incubation and infection), 25 μM forskolin (For), 1 mM and 0.1 mM 8-bromo cyclic AMP (BcA-0.1 and BcA-1, respectively), and 10 μM ionomycin (Jono) and fluorescence was measured using 6-methoxy-1-(3-sulfonatopropyl)quinolinium (SPQ) or N-(ethoxycarbonylmethyl)-6-methoxyquinolinium bromide (MQAE) as chloride-sensitive indicators according to the experiment reported in Example 10.

FIG. 12 shows the inhibitory effect of 50 mM compound IIb (BPIPP) on stimulation of chloride efflux from T84 cells induced with various concentrations of STa from 10 nM to 1 μM according to the experiment reported in Example 11.

### DESCRIPTION OF ILLUSTRATIVE EMBODIMENTS

In one embodiment, the present invention relates to a method of inhibiting activity of adenylyl cyclase or guanylyl cyclase in a mammal by administering to the mammal an amount of a composition effective to inhibit the activity, wherein the composition contains at least one compound selected from the group consisting of structural formulae Ia and Ib and salts thereof:

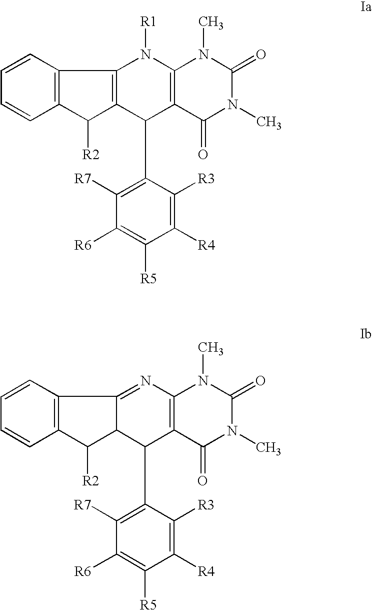

wherein R1 is —H or has the structure —C(═O)R8;

R2 is ═O or has the structure —OC(═O)R9; and

R3, R4, R5, R6, and R7 are each independently selected from the group consisting of —H, —NO₂, -halogen, —OC(═O)R9, —OR9, —OH, —R8OH, —CH₃, —OC(═O)CH₂Ph,

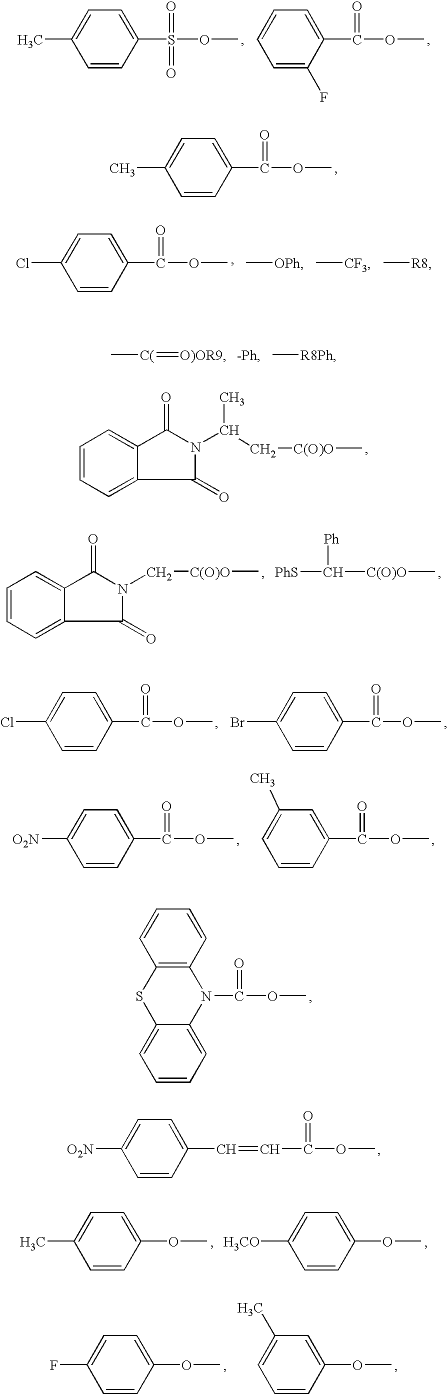

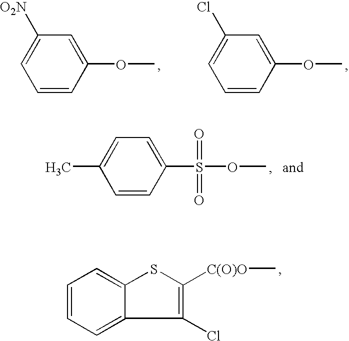

wherein each R8 is independently a linear or branched hydrocarbon group having from 1 to 4 carbon atoms and each R9 is independently a hydrocarbon group having from 1 to 2 carbon atoms.

Any pharmaceutically-acceptable salt of a compound of structural formulae Ia or Ib can be considered as “the salt thereof” Exemplary salts include, but are not limited to, sodium salts, calcium salts, and potassium salts, among others.

“Inhibiting” the activity of adenylyl cyclase or guanylyl cyclase is relative to the activity of the enzyme at the moment prior to administration of the at least one compound selected from the group consisting of structural formulae Ia and Ib and salts thereof. The activity of the enzyme at the moment prior to administration may be higher than, lower than, or at the baseline of enzyme activity seen in the cells of the mammal, the individual mammal, or the mammalian species population.

The at least one compound of structural formulae Ia and Ib and salts thereof can be synthesized by known methods, such as the Mannich condensation reaction (Agarwal, A.; Chauhan, P. M. S., Solid supported synthesis of structurally diverse dihydropyrido[2,3-d]pyrimidines using microwave irradiation. Tetrahedron Letters 2005, 46, (8), 1345-1348; El-Ahl, A. A. S.; El Bialy, S. A. A.; Ismail, M. A., A one-pot synthesis of pyrido[2,3-d]-and quinolino[2,3-d]pyrimidines. Heterocycles 2001, 55, (7), 1315-+; Hagen, H.; Raatz, P.; Walter, H.; Landes, A. Pyrido[2,3-d]pyrimidine-2,4-(1H,3H)-diones, methods for their preparation and herbicides containing them. DE 4035479, 1992; Stankevics, E.; Ozola, A.; Duburs, G., Reaction of 4-aminouracil with arylidene-1,3-indandione. Khimiya geterotsiklicheskikh Soedinenii 1969, (4), 723-6; Stankevics, E.; Popelis, J.; Grinsteins, E.; Ozola, A.; Duburs, G., Constants of the acid dissociation of some nitrogen-containing polynuclear systems. Khimiya geterotsiklicheskikh Soedinenil 1970, (1), 122-4; and Troschutz, R.; Roth, H. J., [Synthesis of pharmacologically active heterocyclic compounds via Mannich reaction, IV: cycloalka(g)- and benzocycloalka(g)pyrido(2,3-d)pyrimidinediones (inventor's transl)]. Arch Pharm (Weinheim) 1978, 311, (6), 542-6).

The composition containing the at least one compound of structural formulae Ia or Ib or salts thereof can contain two or more compounds of structural formulae Ia or Ib.

The at least one compound of structural formulae Ia or Ib or salts thereof may have one or more properties that can make it effective in methods of inhibiting adenylyl cyclase or guanylyl cyclase or methods of treating diseases thereby. Compared to 2-chloroadenosine as described above, the compounds have less or no need for biotransformation and their effects can be detected shortly after administration. Second, the effects of the compounds are generally reversible and they can be removed by treatment with blood or serum or other preparations containing serum albumin. Third, the compounds appear to have low toxicity to cultured cells and do not induce a decrease in the amount of ATP inside the cell. Fourth, particular compounds IIa and IIb generally have a higher potency (typical IC50≈5 μM) than 2-chloroadenosine (IC50≈50 μM). The lower IC50 would be expected to enable the use of lower therapeutic doses of the compounds which suggests, ceteris paribus, fewer systemic effects and lower toxicity. Fifth, the compounds have lower water solubility than 2-chloroadenosine and are generally inactivated by blood or plasma or other fluids of the body containing serum albumin. This suggests there is lower chance that the compounds can be carried to some unintended location and influence the functions of distant parts of the body, again, ceteris paribus, suggesting lower incidence of systemic adverse effects. Sixth, the compounds have not been observed to act on purinergic receptors. Compared to antibiotic treatment of anthrax, the compounds can suppress the function of one of the components of the anthrax toxins, edema toxin, suggesting that the compounds can substantially decrease the lethality associated with anthrax.

In addition to the at least one compound of structural formulae Ia and Ib and salts thereof, the composition can further contain a carrier for pharmaceutical formulations, such as starch, gelatin, or other fillers, binders, or excipients for solid or semisolid formulations such as tablets or capsules, or water, aqueous solution, polar organic solvent, or nonpolar organic solvent for liquid or gel formulations such as orally-dosed liquids, injectable solutions, or topical ointments.

The composition can be in the form of enteric-soluble capsules for oral administration, of aerosol for inhalation for respiratory airway application, of ointment for local application, of cream for local application, of paste for local application, or of solution or solid formulation such as suppositories for rectal administration or solutions for parenteral, intravenous, intrathecal, intramuscular, subcutaneous, or intraperitoneal administration, among others.

The composition can further comprise colorants, flavorants, preservatives, or other inert ingredients known in the art to be suitable in pharmaceutical formulations.

In one further embodiment, the at least one compound has structural formula Ia; R1 has the structure —C(═O)R8; R2 is ═O; R3 is —H; R4 is selected from the group consisting of —H, —NO₂, —Br, —OC(═O)CH₃, and —OR9; R5 is selected from the group consisting of —H, —NO₂, —F, —Cl, and —OC(═O)R9; R6 is —H or —OCH₃; and R7 is —H.

In another further embodiment, the at least one compound has structural formula Ia; R1 is —H; R2 is ═O; R3 is —H; R4 is —Br; R5 is —H; R6 is —H; and R7 is —H. The compound of this embodiment may be referred to herein as “Ia,” 5-(3-bromophenyl)-1,3-dimethyl-5,11-dihydro-1H-indeno[2′,1′:5,6]pyrido[2,3-d]pyrimidine-2,4,6-trione, or BPIPP.

In an additional further embodiment, the at least one compound has structural formula Ib; R2 is —OC(═O)R9; R3 is —H; R4 is —H or —NO₂; R5 is selected from the group consisting of —H, —NO₂, —Br, —Cl, and —OCH₃; R6 is —H; and R7 is —H.

In yet a further embodiment, the at least one compound has structural formula Ib; R2 is ═O; R3 is —H; R4 is —Br; R5 is —H; R6 is —H; and R7 is —H. The compound of this embodiment may be referred to herein as “IIb.”

By inhibiting activity of adenylyl cyclase or guanylyl cyclase in a mammal, administration of the composition described above can, in one embodiment of the present invention, treat a disease in a mammal mediated by activity of adenylyl cyclase or guanylyl cyclase and effected by a toxin produced by a pathogenic organism.

The word “or” is used herein in the inclusive sense.

As used herein, “treating” a disease refers to bringing about any partial or complete abatement of one or more symptoms of the disease, shortening the duration of the disease, or reducing the morbidity or mortality rate resulting from the disease. As part of treatment, the composition can be administered prophylactically or after the onset of symptoms. The composition can be administered on a short-term basis in response to acute symptoms of the disease or prior to an anticipated challenge by a pathogenic microorganism or endogenous disease mechanism, or on a long-term basis in response to chronic symptoms of the disease or prior to an anticipated challenge.

A disease “mediated by activity of adenylyl cyclase or guanylyl cyclase” is used herein to mean a disease in which a reduction in intracellular levels of cyclic AMP or cyclic GMP would reduce the severity, duration, extent, or other parameters of the disease. For example, activation of endogenous enzymes by a toxin of a pathogenic organism (indirect mechanism) can lead to an increase in levels of cAMP or cGMP relative to the levels prior to disease challenge, and reducing cAMP or cGMP levels can reduce the severity, duration, extent, or other parameters of the disease. For another example, infection by a pathogenic organism expressing exogenous enzymes (direct mechanism) can also lead to an increase in levels of cAMP or cGMP relative to levels prior to disease challenge, and reducing cAMP or cGMP levels can reduce the severity, duration, extent, or other parameters of the disease. For a third example, infection by a pathogenic organism or an action by a mechanism endogenous to the mammal can cause a disease which does not involve an increase in the levels of cAMP or cGMP relative to the levels prior to disease challenge, but reducing cAMP or cGMP levels from the levels prior to disease challenge can reduce the severity, duration, extent, or other parameters of the disease.

Compounds known to activate endogenous adenylyl cyclase include, but are not limited to, forskolin and related substances, mastoparan and related substances, peptides simulating the adenylyl cyclase-activating region of the GTP-binding stimulatory protein (Gs), other agents acting through receptor-independent activation of the Gs including cholera toxin, labile toxin LT from Escherichia coli, activators of ADP-ribosylation of Gs and inhibitors of deADP-ribosylation of Gs as well as various hormones, mediators, synthetic compounds, and other similar agents which are stimulating adenylyl cyclase through activation of Gs-coupled receptors and other agents including antagonists of the GTP-binding inhibitory protein (Gi) such as islet-activating protein pertussis toxin, inhibitors of Gi effect on adenylyl cyclase and also various hormones, mediators, and synthetic compounds which suppress the Gi function in the cell.

Compounds known to activate endogenous guanylyl cyclase include, but are not limited to, nitric oxide and nitric oxide donors, allosteric and indirect activators of soluble guanylyl cyclase including substances which bind directly to guanylyl cyclase and substances which increase activity of guanylyl cyclase indirectly by increasing the sensitivity of guanylyl cyclase to stimulation with nitric oxide, or substances which decrease desensitization of guanylyl cyclase induced by nitric oxide, or substances which increase the amount of nitric oxide in cells and tissues without producing nitric oxide by themselves but by activating synthesis of nitric oxide in cells and tissues, or inducing release of nitric oxide from endogenous stores in the cells or tissues, or increasing production of nitric oxide from either nitric oxide generating substances or by increasing stability of nitric oxide which was generated from nitric oxide generating substances or from endogenous sources such as enzyme nitric oxide synthase or other endogenous stores of nitric oxide thus increasing the effective amount of nitric oxide in cells or tissues; also including other stimulators of guanylyl cyclase such as various hormones and mediators acting by directly binding to guanylyl cyclase including atrial natriuretic peptide, brain natriuretic peptide, C-type natriuretic peptide, guanylin, uroguanylin, lymphoguanylin and bacterial toxins and their analogs and other substances which either increase sensitivity of guanylyl cyclase to stimulation with direct stimulators or which decrease desensitization of guanylyl cyclase when being stimulated by the direct stimulators.

The method can treat diseases involving the direct mechanism by inhibiting bacterial adenylyl cyclase toxins in cells and tissues such as adenylyl cyclase toxins of Bordetella pertussis and Bordetella parapertussis and other similar toxins of Bordetella spp., Exo Y toxin of Pseudomonas aeruginosa and other similar toxins of representatives of the bacterial family Pseudomonadaceae, adenylyl cyclase of Yersinia pestis and other similar proteins of the Yersinia spp., and adenylyl cyclase (edema factor) which is the component of the edema toxin of Bacillus anthracis.

In one embodiment, the pathogenic organism is selected from the group consisting of Escherichia coli, Vibrio cholerae, Bordetella spp., Pseudomonas spp., Yersinia spp., and Bacillus anthracis. In a further embodiment, the Bordetella species can be Bordetella pertussis or Bordetella parapertussis. In a further embodiment, the Pseudomonas species can be Pseudomonas aeruginosa. In a further embodiment, the Yersinia species can be Yersinia pestis.

In one embodiment, the disease is selected from the group consisting of diarrhea, cholera, pertussis (whooping cough), and anthrax. In a further embodiment, the method can be used for treatment of various types of diarrhea which involve activation of cyclic AMP and cyclic GMP synthesis in intestinal brush border or other cells. The diarrhea can be treated in human (Homo sapiens) patients and in various animals including cattle (Bos spp.) and swine (Sus spp.). In another embodiment, the main pathogen of the diarrhea is an enterotoxigenic bacterium selected from the group consisting of Escherichia coli, Campylobacter, Shigella, Salmonella, Bacillus aureus, Staphylococcus aureus, Vibrio cholerae, Clostridium perfringens, Clostridium difficile, Klebsiella pneumoniae, Aeromonas, Vibrio parahaemolyticus. In another embodiment, the diarrhea presents in a patient with acute hepatitis A or B or in a patient with immunodeficiency.

When the disease is diarrhea, the composition can be in the form of an enteric-coated capsule.

In one embodiment, the compound of structural formulae Ia or Ib or salts thereof can be administered to the mammal at dose of 0.1 mg-1000 mg/kg body weight per 4 hours.

Various pharmaceutical compositions having the substances described in the present invention as active ingredients can be prepared by methods previously described in the art for active ingredients poorly soluble in water. For example, the following method can be used to prepare enteric-coated tablets containing compound IIb as an active ingredient.

The tablet ingredients are dry-blended including (weight percentage) compound IIb (80%), microcrystalline cellulose (10%), corn starch (5%), croscarmellose sodium (4%), syloid (0.5%), and stearic acid (0.5%). Then the tablet is prepared by pressing (weight 500 mg). The tablets are then coated in 10% coating solution containing (weight percentage) Eastacryl 300 (64.4%), water (28.8%), triethyl citrate (1.9%), talc (4.7%), and an antifoam such as Dow Corning 1520-US (0.2%) using a side-vent pan coater by the standard methods known in the art.

The active ingredient would then be insoluble in the stomach but the coating would dissolve in intestine and thus the active ingredient can be delivered to the intestine for treatment of diarrhea. Other pharmaceutical compositions can be prepared by methods known in the art.

In one embodiment when the disease is diarrhea, intestinal-soluble capsules can be used. Compound IIb can be used at a dose of 10-20 mg/kg body weight per 4 hour period. For example, if a tablet contains 400 mg of the active ingredient, this means that the dose is 8-18 tablets per 24 hour period administered at starting from 2 tablets every 6 hours to 3 tablets every 4 hours for adult otherwise healthy patients.

When the disease is pertussis, the composition can be in the form of an inhalable aerosol.

When the disease is anthrax, the composition can be in a form suitable for the particular site of the anthrax. To treat cutaneous anthrax, the composition can be in the form of an ointment, cream, or paste for local application. To treat inhalational anthrax, the composition can be in the form of an aerosol for inhalation for respiratory airway application. To treat gastrointestinal anthrax, the composition can be in the form of an enteric-coated capsule for oral administration or a solution or solid formulation for rectal administration.

In one embodiment when the disease is anthrax, compound IIb can be used at a dose of 10-20 mg/kg body weight per 4 hour period. For example, if a tablet contains 400 mg of compound IIb, this means that the dose is 8-18 tablets per 24 hour period administered at starting from 2 tablets every 6 hours to 3 tablets every 4 hours for adult otherwise healthy patients.

In treating anthrax, a method according to the present invention can be performed in conjunction with known methods. It is known in the art to treat anthrax with penicillin G at 2 million units at 6 hour intervals until edema is decreased with subsequent administration of oral penicillin to complete a 7-10-day course in cutaneous form. In case of penicillin sensitivity, treatment with ciprofloxacin, erythromycin, tetracycline, or chloroamphenicol can be substituted. In case of inhalational or gastrointestinal anthrax, penicillin is used at high doses (8-12 million units per day in divided doses at 4-6 h intervals).

In one embodiment, the method can involve using at least one compound having formula IIa or formula IIb for inhibition of cyclic AMP increase induced by cholera toxin of Vibrio cholera and labile toxin LT of E. coli and by adenylyl cyclase toxin of Bordetella pertussis and edema factor of Bacillus anthracis and for inhibition of cyclic GMP increase induced by stable toxin ST of E. coli or guanylin or other related peptides in cells and tissues.

By inhibiting activity of adenylyl cyclase or guanylyl cyclase in a mammal, administration of the composition described above, in one embodiment of the present invention, can reduce cyclic AMP or cyclic GMP levels in a mammal in need of such reduction. The need can arise as a result of or in conjunction with a disease effected by a mechanism endogenous to the mammal.

This method can reduce the effects of various hormones, growth factors, cytokines, peptides, neurotransmitters, autocrine or paracrine substances, mediators, and other natural or synthetic agents whose effects are mediated through increased levels of cyclic AMP and cyclic GMP.

In another embodiment, the disease is selected from the group consisting of cystic fibrosis, endocrinopathies, chronic obstructive pulmonary disease, adrenal cancer, pituitary cancer, lung cancer, chromaffin tumor, and parathyroid tumor.

In another embodiment, the disease is chronic diarrhea in children and adults wherein the diarrhea is induced by secretory causes, by various types of medications, by bowel resection, by mucosal disease, by enterocolic fistula, by various type of hormone disbalance, by congenital defects in ion absorption, by inflammatory causes including inflammatory bowel disease, microscopic colitis, collagenous colitis, food allergy, eosinophilic gastroenteritis, and graft-versus-host disease, by radiation injury, by gastrointestinal malignancies, by pancreatitis, by steatorrhea, and by dismotility causes (such as visceral neuromyopathies, hyperthyroidism, and induced by prokinetic agents). In this embodiment, the composition can be in the form of an enteric-coated capsule.

In any embodiment of treating a disease according to the present invention, treatment can further involve administering to the mammal one or more inhibitors of adenylyl cyclase or guanylyl cyclase other than the at least one compound described above. Examples of inhibitors of adenylyl cyclase include, but are not limited to, inhibitors of the catalytic component of adenylyl cyclase, inhibitors of Gs coupling to adenylyl cyclase and various inhibitors of hormone-dependent stimulation of adenylyl cyclase including antagonists, partial antagonists, and blockers of receptors which stimulate adenylyl cyclase and other agents which diminish hormone-dependent stimulation of adenylyl cyclase by interference with intracellular signaling pathways or extracellular signaling pathways or with hormone production, degradation, or stability.

Examples of inhibitors of guanylyl cyclase include, but are not limited to, inhibitors of soluble guanylyl cyclase or inhibitors of nitric oxide binding to soluble guanylyl cyclase, or agents which oxidize the heme iron of the soluble guanylyl cyclase, or inhibitors of nitric oxide production, or substances which induce trapping or accelerate decomposition of nitric oxide thereby decreasing the levels of nitric oxide; and also including inhibitors of hormone dependent stimulation of guanylyl cyclase including, but not limited to, antagonists, partial antagonists, and blockers of receptors which stimulate guanylyl cyclase and other agents which diminish hormone-dependent stimulation of guanylyl cyclase by interference with intracellular signaling pathways or extracellular signaling pathways or with hormone production, degradation, or stability.

In one embodiment of treating a disease according to the present invention, administration of the composition can suppress stimulation of intestinal or epithelial ion transport induced in intestinal mucosa brush border cells or airway epithelial cells or other barrier epithelial cells by various agents which may act by increasing the levels of cAMP or cGMP in these cells, including bacterial toxins and other endogenous and exogenous, natural or synthetic, agents. In a further embodiment, the method can involve administration of at least one compound having formula Ia or formula IIb can inhibit chloride ion transport in intestinal mucosa brush border cells induced by cholera toxin of Vibrio cholera and labile toxin LT and stable toxin ST of E. coli.

In any embodiment of treating a disease according to the present invention, treatment can be directed against disorders associated with increased epithelial permeability, including protection of airway epithelium integrity compromised with bacterial infection (such as Pseudomonas aeruginosa) or resulting from cystic fibrosis or chronic obstructive pulmonary disease or gastrointestinal disorders including, but not limited to, ulcerative colitis, regional enteritis, or inflammatory bowel disease.

In one embodiment, the compound of structural formulae Ia or Ib or salts thereof can be administered to the mammal at dose of 0.1 mg-1000 mg/kg body weight per 4 hours.

In another embodiment, the present invention relates to a method of inhibiting activity of adenylyl cyclase or guanylyl cyclase in mammalian cells in vitro by administering to the mammalian cells an amount of a composition effective to inhibit the activity, wherein the composition contains at least one compound selected from the group consisting of structural formulae Ia and Ib and salts thereof.

The at least one compound and the composition can be as described above. The mammalian cells can be cells of any cell line of any mammalian species. Such cells can be prepared and maintained by techniques known in the art. In one embodiment, the mammalian cells are selected from the group consisting of T84 human colonic carcinoma cells and rat lung fibroblast RFL-6 cells.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Examples

### 1. Compounds Tested.

The compounds were purchased from ChemDiv, Inc., San Diego, Calif. Compounds of interest are shown in Table 1.

TABLE 1

| ID | Formula |
|---|---|
| IIa | 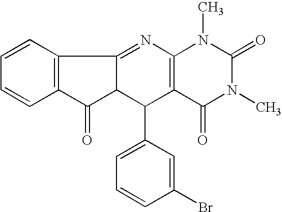 |
| IIb | 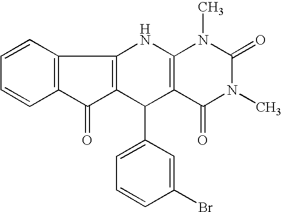 |
| IIIa | 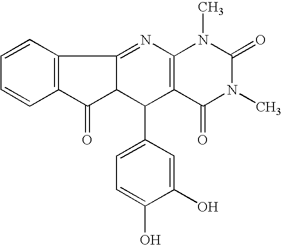 |
| IIIb | 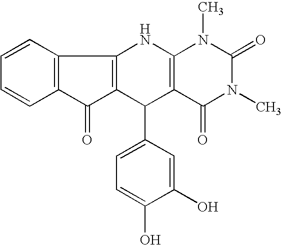 |
| IVb | 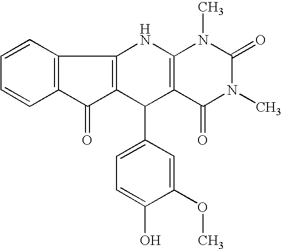 |
| Vb | 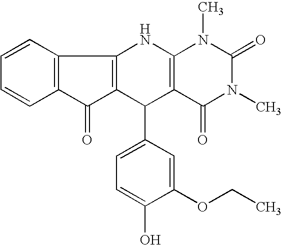 |
| VI | 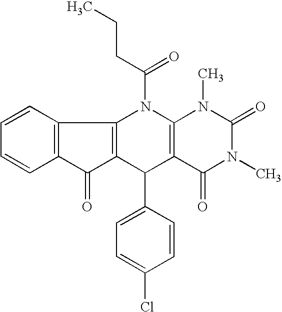 |
| VII | 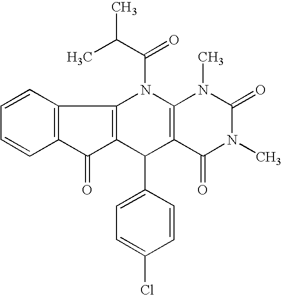 |

### 2. Inhibition of STa-Induced Activation of GC-C in T84 Cells.

T84 cells were grown in 12-well plates to confluency at 37° C. in a humidified atmosphere containing 5% CO₂ and the medium was replaced with 0.5 ml of phosphate buffered Dulbecco's solution (DPBS) containing 1 mM 3-isobutyl-1-methyl xanthine (IBMX) and vehicle dimethylsulfoxide (DMSO) at concentration 0.1% v/v or containing compound IIb at concentration 50 μM. Cells were incubated for 10 min at 37° C. and treated with or without STa (100 nM final concentration). After 10 min incubation, medium was aspirated and cyclic GMP was extracted by addition of 0.3 ml 50 mM sodium acetate buffer, pH 4.0, and rapid freezing at −80° C. Plate was thawed and contents of cyclic GMP were assayed in the extract using enzyme-linked immunosorbent assay developed as described in the art (Horton, J. K.; Martin, R. C.; Kalinka, S.; Cushing, A.; Kitcher, J. P.; O'Sullivan, M. J.; Baxendale, P. M., Enzyme immunoassays for the estimation of adenosine 3′,5′ cyclic monophosphate and guanosine 3′,5′ cyclic monophosphate in biological fluids. J Immunol Methods 1992, 155, (1), 31-40). The pellet of cells was used to assay protein contents. The data are expressed in pmol of cGMP accumulated per mg of protein.

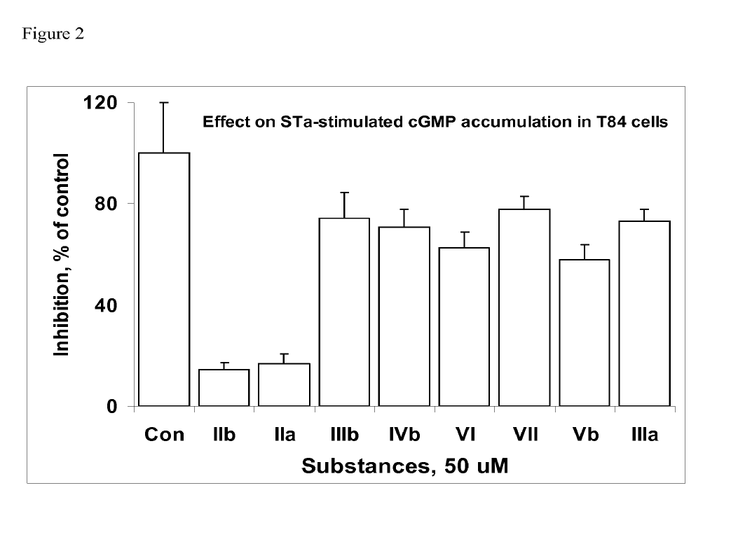

Basal contents of cyclic GMP in untreated cells was 6±2 pmol/mg protein. When cells were treated with 100 nM STa in the presence of vehicle (0.1% DMSO), the level of cyclic GMP was 124±10 pmol/mg protein. When cells were treated by STa in the presence of 50 μM compounds IIa or IIb, the levels of cyclic GMP were 21±4 or 24±6 pmol/mg protein, respectively (inhibition by 87.1% and 84.7%). The data obtained with other compounds of the invention are summarized in FIG. 2.

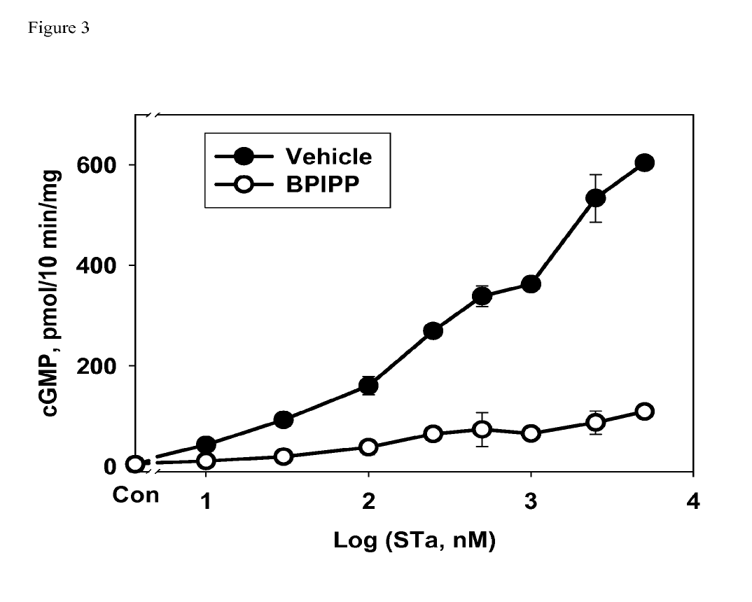

When T84 cells were treated in the presence of vehicle (0.1% DMSO), increasing concentrations of STa induced progressively increasing accumulation of cyclic GMP. In T84 cells treated with STa in the presence of 50 μM compound IIb (BPIPP), this increase was significantly suppressed. Results of a representative experiment are shown in FIG. 3.

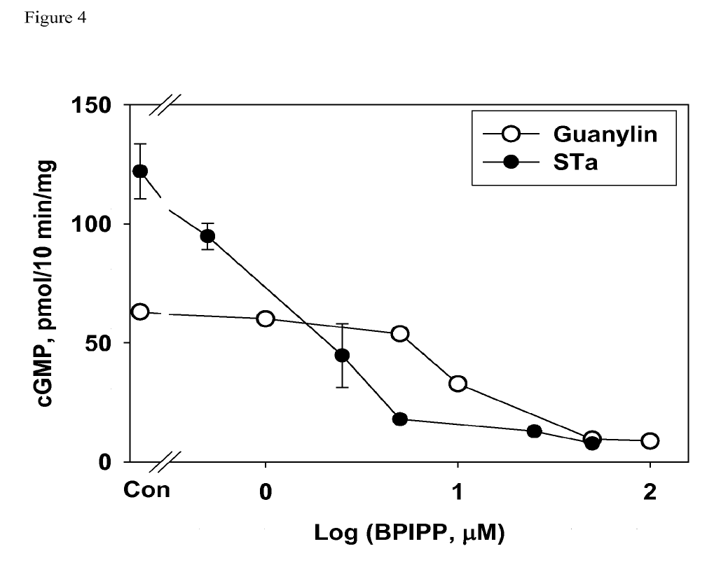

When T84 cells were treated with 100 nM STa in the presence of various concentrations of compound IIb (BPIPP), increasing concentrations of compound IIb produced stronger inhibition of cyclic GMP synthesis in the stimulated cells. Results of a representative experiment are shown in FIG. 4. The value of IC50 for compound IIb in this experiment was 3.4 μM.

These data indicate that the compounds can inhibit the STa-stimulated increase in cyclic GMP accumulation.

### 3. Inhibition of Guanylin-Induced Activation of GC-C in T84 Cells.

This experiment followed the protocols described in Example 2. When cells were treated with 500 nM guanylin in the presence of vehicle (0.1% DMSO), the level of cyclic GMP was 124±13 pmol/mg protein. When cells were treated with guanylin in the presence of 50 μM compounds Ia or IIb, the level of cyclic GMP was 124±13, 23±12, or 51±6 pmol/mg protein, respectively (81.4% and 58.9% inhibition, respectively).

When T84 cells were treated with 500 nM guanylin in the presence of various concentrations of compound IIb (BPIPP), increasing concentrations of compound IIb produced stronger inhibition of cyclic GMP synthesis in the stimulated cells. Results of a representative experiment are shown in FIG. 4. The value of IC50 for compound IIb in this experiment was 7.2 μM.

These data indicate that at least compounds Ia and IIb can inhibit guanylin-stimulated increase in cyclic GMP accumulation.

### 4. Inhibition of Natriuretic Peptide- and Nitric Oxide-Induced Activation of Cyclic GMP Accumulation in Various Cell Types.

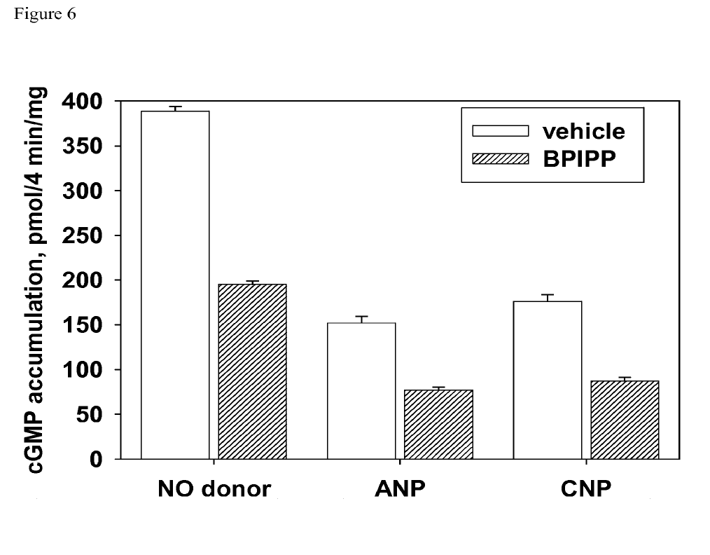

This experiment followed the protocols described in Example 2. The compound Ia (50 μM) inhibited cyclic GMP accumulation in a neuroblastoma cells line BE-2(C) stimulated with a nitric oxide donor, benzotrifuroxan (10 μM), by 65.4%. Similar results were obtained in rat RFL-6 fibroblast cells stimulated with nitric oxide donors (10 μM benzotrifuroxan and diethylamine/NONOate), 1 μM atrial natriuretic peptide (ANP) and C-type natriuretic peptide (CNP) with inhibition by 50.2%, 50.6%, and 49.4%, respectively, and in BE-2(C) cells stimulated with ANP and CNP with inhibition by 62.6% and 85.9%, respectively. Results of a representative experiment with similar setup but using compound IIb (BPIPP) are shown in FIG. 6.

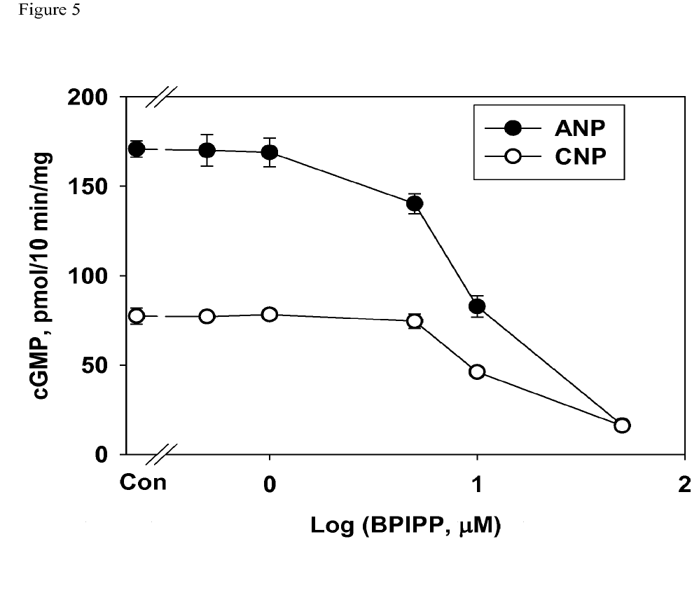

When BE-2 cells were treated with 1 μM ANP and 0.5 μM CNP in the presence of various concentrations of compound IIb (BPIPP), increasing concentrations of compound IIb produced stronger inhibition of cyclic GMP synthesis in the stimulated cells. Results of a representative experiment are shown in FIG. 5. The value of IC50 for compound IIb in this experiment was 8.4 μM in the case of ANP-induced elevation in intracellular cyclic GMP contents and 11.2 μM in the case of CNP-induced elevation in intracellular cyclic GMP contents.

These data indicate that at least compounds Ia and IIb can inhibit natriuretic peptide- and nitric oxide-induced activation of cyclic GMP accumulation in various cells and tissues.

### 5. Inhibition of Cholera Toxin-Induced Activity of Adenylyl Cyclase in T84 Cells.

This experiment followed the protocols described in Example 2, but the amount of cyclic AMP accumulated was assayed with a commercially available kit from Cayman Chemical Co., Ann Arbor, Mich.

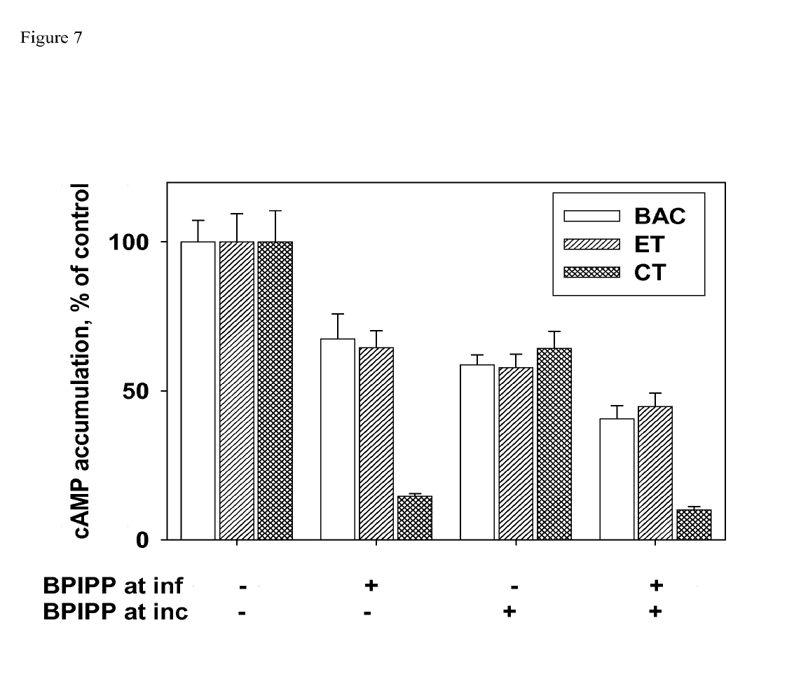

When T84 cells were treated with cholera toxin (1 μg/ml) for 60 min (infection phase) in the presence of vehicle DMSO (0.1%) in serum-free medium and then for additional 10 min (incubation phase) in the presence of vehicle and 1 mM IBMX in DPBS, the amount of cyclic AMP accumulated in the cells was 7.8±0.8 nmol/mg protein. When the cells were treated with cholera toxin in the presence of 50 μM compound IIb (BPIPP), the amount of cyclic AMP accumulated in the cells was 1.2±0.1 nmol/mg protein (84.6% inhibition). When 50 μM compound IIb was present during both infection and incubation phases, the inhibition of cholera toxin effects was even more pronounced. Results of a representative experiment are shown in FIG. 7.

These data indicate that at least compound IIb can inhibit the activity of cholera toxin in intestinal secretory epithelium.

### 6. Inhibition of Various Types of Activators of Adenylyl Cyclase in Various Cell Types.

This experiment followed the protocols described in Example 2, but the amount of cyclic AMP accumulated was assayed with a commercially available kit from Cayman Chemical Co.

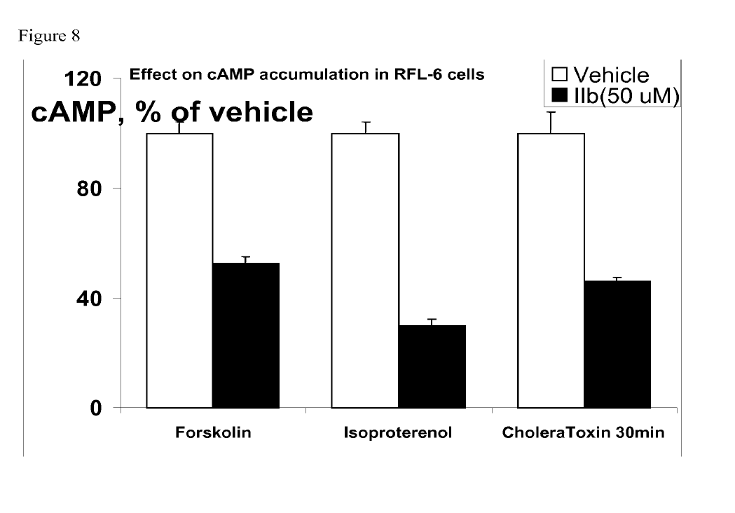

In T84 cells, 50 μM compound IIb inhibited activation of cyclic AMP accumulation induced by treatment with 10 μM forskolin (by 41.3%) and 100 μM isoproterenol (by 58.2%). Similar results were obtained in rat RFL-6 cells treated with 10 μM forskolin, 100 μM isoproterenol, and 2 μg/ml cholera toxin for 30 min (FIG. 8).

These data indicate that at least compound IIb can inhibit stimulation of adenylyl cyclase by various agents in various cells and tissues.

### 7. Lack of Effect of Intracellular ATP Levels.

This experiment followed the protocols described in Example 2, but ATP levels were determined by using the commercially available “ATPLite” kit from Perkin Elmer, Wellesley, Mass., and described by http://las.perkinelmer.com/content/Manuals/BookletATPlite.pdf (accessed Jun. 21, 2006).

Treatment of T84 cells with 50 μM compounds Ia and IIb did not change the intracellular ATP level compared to cell treated with vehicle (0.1% DMSO) or untreated cells. The corresponding ATP contents were 18.3±1.5; 16.4±1.7; 22.2±4.3; 20.3±2.4 nmol/mg protein, respectively.

These data indicate that at least compounds Ia and IIb do not influence the level of intracellular ATP.

### 8. Lack of Effect on Degradation of Cyclic Nucleotides.

Degradation of cGMP was assayed in the extracts of T84 cells treated with vehicle DMSO (0.1%) or 50 μM compound IIb. Cyclic GMP (10 μM) was added to the extracts, incubated for 10 min, and the amount of cyclic GMP remaining was measured as described in Example 2.

The rate of cyclic GMP disappearance was identical in all extracts prepared and compound IIb did not statistically significantly influence this rate (21.8±0.8 nmol/min in control and 25.7±2.3 nmol/min in treatment groups). N=3.

These data indicate that at least compound IIb does not influence degradation of cyclic nucleotides.

### 9. Lack of Effect on Extrusion of Cyclic Nucleotides.

This experiment followed the protocols described in Example 2, using T84 cells.

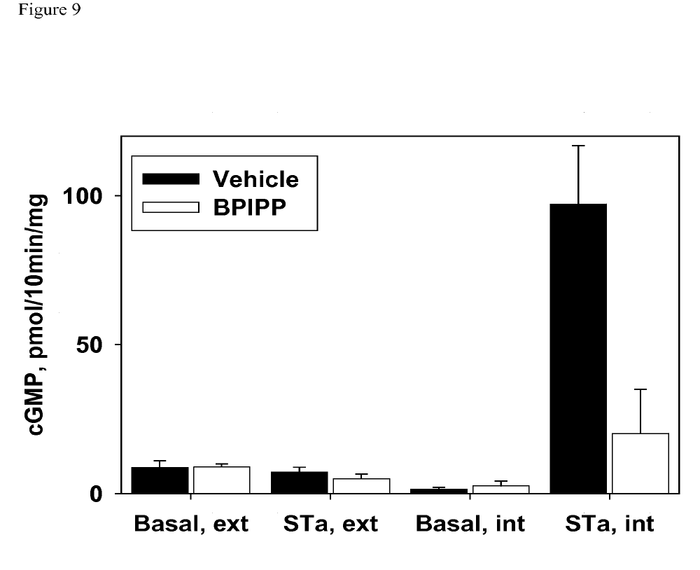

Extracellular amounts of cyclic GMP were measured in the medium of cells treated with 100 nM STa (7.3±1.6 pmol/mg protein) and with STa in the presence of 50 μM compound IIb (BPIPP; 5.1±1.5 pmol/mg protein). N=3. Results of a representative experiment are shown in FIG. 9.

These data indicate that at least compound IIb does not influence extrusion of cyclic nucleotides to the extracellular medium.

### 10. Inhibition of Adenylyl Cyclase-Dependent Chloride Transport in T84 Cells.

This experiment followed the protocols described in Example 2. The chloride transport assay was performed by a method known in the art (West, M. R.; Molloy, C. R., A microplate assay measuring chloride ion channel activity. Anal Biochem 1996, 241, (1), 51-8). In some experiments, MQAE was used as a fluorescent sensor for chloride anions and in other experiments, SPQ was used to detect chloride efflux from T84 cells. Both experimental approaches gave similar results.

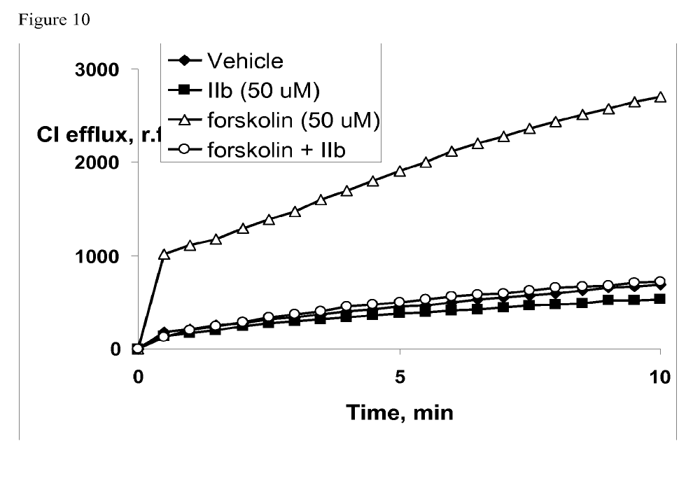

Treatment of T84 cells with 50 μM forskolin significantly increased the rate of chloride anion efflux from the cells measured as increase in fluorescence of the intracellular dye up to 2690 relative units in 10 min from the baseline of 477 relative units in 10 min. Pretreatment of T84 cells with forskolin and 50 μM compound IIb decreased the response down to 428 relative units in 10 min. The dye used in the assay, N-(ethoxycarbonylmethyl)-6-methoxyquinolinium bromide (MQAE), was a specific reagent to detect intracellular chloride ions. Results of a representative experiment are illustrated in FIG. 10.

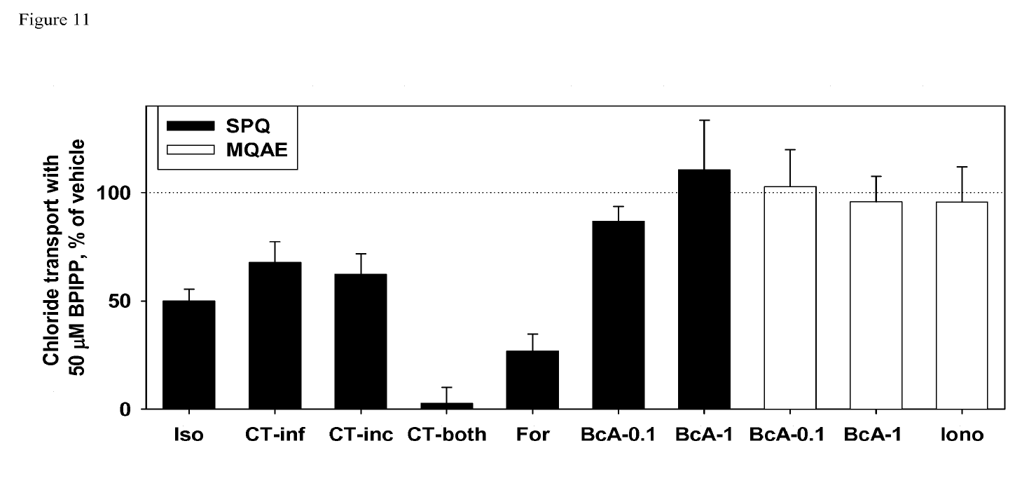

When T84 cells were treated with isoproterenol, cholera toxin, forskolin (activators of adenylyl cyclase), a cell-permeable cyclic AMP analog 8-bromo cyclic AMP, or a calcium ionophore ionomycin, chloride efflux from the cells was increased. In cells treated with isopoterenol, cholera toxin, and forskolin in the presence of 50 μM compound IIb, this increase in chloride efflux was significantly suppressed. However, increase in chloride efflux induced by 8-bromo cyclic AMP or inomycin was not influenced by compound IIb. Results of a representative experiment are illustrated in FIG. 11.

These data indicate that at least compound IIb can inhibit cyclic AMP-stimulated chloride anion transport in cells and tissues associated with activation of adenylyl cyclase.

### 11. Inhibition of STa-Induced Chloride Transport in T84 Cells.

This experiment followed the protocol described in Examples 2 and 10. SPQ was used to detect chloride efflux from T84 cells.

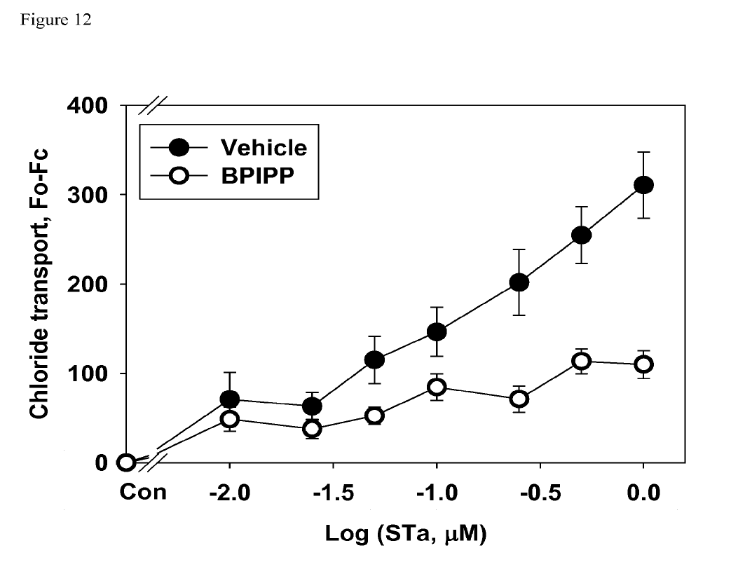

Treatment of T84 cells with increasing concentrations of STa in the presence of vehicle (0.1% DMSO) progressively increased chloride efflux from the cells. When cells were treated with 50 μM compound IIb (BPIPPO, this increase was significantly suppressed. Results of a representative experiment are illustrated in FIG. 12.

These data indicate that at least compound IIb can inhibit cyclic GMP-stimulated chloride anion transport in cells and tissues associated with activation of guanylyl cyclase.

### 12. Inhibition of STa- and Forskolin-Induced Increase in Short Circuit Current in T84 Cells.

This experiment followed the protocols described in Example 2. The assay was performed by the method described by Hug at http://pen2.igc.gulbenkian.pt/cftr/vr/d/hug_transepithelial_measurements_using_the_ussing_chamber.pdf (accessed Jun. 21, 2006). T84 cells were grown on a Millipore nitrocellulose filter support inserts which were mounted in a standard Ussing chamber system. Chambers were filled with a Krebs bicarbonate solutions containing (in mM) NaCl (115), KCl (4.7), MgCl₂ (1.13), NaHCO₃ (25), Na₂HPO₄ (1.15), glucose (10), and CaCl₂ (1), pH 7.4, at 37° C. and constantly bubbled with carbogen. Short circuit current was recorded using a voltage clamp (2-6 mV) with Ag/AgCl electrodes and agar bridges.

When cell monolayers were treated with DMSO vehicle (0.1%) or with 50 μM compound Ia, there was no change in current. When cells were treated with 100 nM STa, the background current was increased by 9.6±1.3 μA/cm². DMSO vehicle did not influence this value. When STa and compound Ia were added together, current increased only by 2.7±0.8 μA/cm2 (inhibition by 71.9%). Forskolin at 50 μM increased the current to 17.6 μA/cm² and forskolin in the presence of compound Ia increased the current only by 4.9 μA/cm² (inhibition by 72.2%). N=2-4.

These data indicate that at least compound Ia can inhibit ion transport stimulated by increased cyclic nucleotide accumulation.

### 13. Inhibition of Liquid Secretion Induced by STa in a Rabbit Intestinal Loop Model.

This experiment followed the protocols described in Example 2. The test was performed by the method known in the art (Alcantara, C. S.; Jin, X. H.; Brito, G. A.; Carneiro-Filho, B. A.; Barrett, L. J.; Carey, R. M.; Guerrant, R. L., Angiotensin II subtype 1 receptor blockade inhibits Clostridium difficile toxin A-induced intestinal secretion in a rabbit model. J Infect Dis 2005, 191, (12), 2090-6). Volume of liquid in the loop was divided by the length of the loop and the normalized value was used to assess liquid accumulation in the intestinal lumen.

When loops were injected with 100 nM STa and vehicle DMSO (0.01%), liquid accumulation was 0.62±0.05 ml/cm. When the loops were injected with STa and 50 μM compound IIb, liquid accumulation was 0.15±0.06 ml/cm. In loops injected with phosphate buffered saline (vehicle for STa), liquid accumulation was 0.18±0.03 ml/cm. Compound IIb was inhibiting STa-stimulated liquid accumulation completely. N=2-6.

These data indicate that at least compound IIb can be used in vivo for treatment of diarrhea induced by bacterial infection.

14. Inhibition of Cyclic AMP Accumulation in Cells Infected with Pertussis Adenylyl Cyclase Toxin.

The assay was performed in T84 cells by the methods described in Example 2 and Ahuja, N.; Kumar, P.; Bhatnagar, R., The adenylate cyclase toxins. Crit Rev Microbiol 2004, 30, (3), 187-96, but the amount of cyclic AMP accumulated was assayed with a commercially available kit from Cayman Chemical Co.

Cells were treated with pertussis adenylyl cyclase toxin at 1 μg/ml in Dulbecco's minimal essential medium for 60 min in the presence of vehicle DMSO (0.1%) or 50 μM compound IIb for 60 min and then medium was aspirated and replaced with DPBS containing 1 mM IBMX and DMSO or compound IIb, respectively, for the vehicle and compound IIb-treated samples. After 10 min incubation, the amount of cyclic AMP was measured. In the samples containing vehicle, cyclic AMP accumulation was 9.97±0.72 nmol/mg protein and compound IIb decreased it to 4.06±0.44 nmol/mg protein (by 59.2%). N=3. Results of a representative experiment are shown in FIG. 7.

These data indicate that at least compound IIb can inhibit the activity of pertussis adenylate cyclase toxin.

15. Inhibition of Cyclic AMP Accumulation in Cells Infected with Anthrax Edema Toxin.

The assay was performed in T84 cells by the methods described in Example 2 and Leppla, S. H., Anthrax toxin edema factor: a bacterial adenylate cyclase that increases cyclic AMP concentrations of eukaryotic cells. Proc Natl Acad Sci USA 1982, 79, (10), 3162-6, but the amount of cyclic AMP accumulated was assayed with a commercially available kit from Cayman Chemical Co.

Cells were treated with protective antigen 2 μg/ml and edema factor 0.1 μg/ml (forming edema toxin (Mourez, M., Anthrax toxins. Rev Physiol Biochem Pharmacol 2004, 152, 135-64)) in Dulbecco's minimal essential medium for 60 min in the presence of vehicle DMSO (0.1%) or 50 μM compound IIb for 60 min and then medium was aspirated and replaced with DPBS containing 1 mM IBMX and DMSO or compound Ia, respectively, for the vehicle and compound IIb-treated samples. After 10 min incubation, the amount of cyclic AMP was measured. In the samples containing vehicle, cyclic AMP accumulation was 138.5±13.1 pmol/mg protein and compound IIb decreased it to 62.1±6.3 pmol/mg protein (by 55.2%). N=3. Results of a representative experiment are shown in FIG. 7.

These data indicate that at least compound IIb can inhibit the activity of anthrax edema toxin.

### 16. Inhibition of Growth of Tumor Cells.

The colorimetric 3-(4,5-dimethylthiazole-2-yl)-2,5-diphenylformazan assay essentially as described by Cario E.; Goebell H.; Dignass A. U., Factor XIII modulates intestinal epithelial wound healing in vitro. Scand. J. Gastroenterol. 1999, 34, (5), 485-90, was used to evaluate the effect of compound Ia on growth of tumor cells.

T84 colonic carcinoma cells were treated for 24 h in serum-free medium in the presence of vehicle (0.1% DMSO) or variable concentrations of compound Ia (from 1 to 50 μM). Growth of T84 cells was not affected by concentrations of up to 5 μM. At 10 μM concentration, growth was inhibited by 30.8% and at 50 μM concentration, growth was inhibited by 38.7%. N=8.

These data indicate that at least compound IIa can inhibit the growth of tumor cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Parkinson, S. J.; Alekseev, A. E.; Gomez, L. A.; Wagner, F.; Terzic, A.; Waldman, S. A., Interruption of Escherichia coli heat-stable enterotoxin-induced guanylyl cyclase signaling and associated chloride current in human intestinal cells by 2-chloroadenosine. J Biol Chem 1997, 272, (2), 754-8.
2. Agarwal, A.; Chauhan, P. M. S., Solid supported synthesis of structurally diverse dihydropyrido[2,3-d]pyrimidines using microwave irradiation. Tetrahedron Letters 2005, 46, (8), 1345-1348.
3. El-Ahl, A. A. S.; El Bialy, S. A. A.; Ismail, M. A., A one-pot synthesis of pyrido[2,3-d]- and quinolino[2,3-d]pyrimidines. Heterocycles 2001, 55, (7), 1315-+.
4. Hagen, H.; Raatz, P.; Walter, H.; Landes, A. Pyrido[2,3-d]pyrimidine-2,4-(1H,3H)-diones, methods for their preparation and herbicides containing them. DE 4035479, 1992.
5. Stankevics, E.; Ozola, A.; Duburs, G., Reaction of 4-aminouracil with arylidene-1,3-indandione. Khimiya geterotsiklicheskikh Soedinenii 1969, (4), 723-6.
6. Stankevics, E.; Popelis, J.; Grinsteins, E.; Ozola, A.; Duburs, G., Constants of the acid dissociation of some nitrogen-containing polynuclear systems. Khimiya geterotsiklicheskikh Soedinenii 1970, (1), 122-4.
7. Troschutz, R.; Roth, H. J., [Synthesis of pharmacologically active heterocyclic compounds via Mannich reaction, IV: cycloalka(g)- and benzocycloalka(g)pyrido(2,3-d)pyrimidinediones (author's transl)]. Arch Pharm (Weinheim) 1978, 311, (6), 542-6.
8. Horton, J. K.; Martin, R. C.; Kalinka, S.; Cushing, A.; Kitcher, J. P.; O'Sullivan, M. J.; Baxendale, P. M., Enzyme immunoassays for the estimation of adenosine 3′,5′ cyclic monophosphate and guanosine 3′,5′ cyclic monophosphate in biological fluids. J Immunol Methods 1992, 155, (1), 31-40.
9. West, M. R.; Molloy, C. R., A microplate assay measuring chloride ion channel activity. Anal Biochem 1996, 241, (1), 51-8.
10. Alcantara, C. S.; Jin, X. H.; Brito, G. A.; Cameiro-Filho, B. A.; Barrett, L. J.; Carey, R. M.; Guerrant, R. L., Angiotensin II subtype 1 receptor blockade inhibits Clostridium difficile toxin A-induced intestinal secretion in a rabbit model. J Infect Dis 2005, 191, (12), 2090-6.
11. Ahuja, N.; Kumar, P.; Bhatnagar, R., The adenylate cyclase toxins. Crit Rev Microbiol 2004, 30, (3), 187-96.
12. Leppla, S. H., Anthrax toxin edema factor: a bacterial adenylate cyclase that increases cyclic AMP concentrations of eukaryotic cells. Proc Natl Acad Sci USA 1982, 79, (10), 3162-6.
13. Mourez, M., Anthrax toxins. Rev Physiol Biochem Pharmacol 2004, 152, 135-64.
14. http://las.perkinelmer.com/content/Manuals/BookletATPlite.pdf (accessed Jun. 21, 2006)
15. Hug, http://pen2.igc.gulbenkian.pt/cftr/vr/d/hug_transepithelial_measurements_using_the_ussing_chamber.pdf (accessed Jun. 21, 2006).
16. Cario E.; Goebell H.; Dignass A. U., Factor XIII modulates intestinal epithelial wound healing in vitro. Scand. J. Gastroenterol. 1999, 34, (5), 485-90.

What is claimed is:

1. A method of inhibiting activity of adenylyl cyclase or guanylyl cyclase in a mammal, comprising:
administering to the mammal an amount of a composition effective to inhibit the activity, wherein the composition comprises at least one compound selected from the group consisting of structural formulae Ia and Ib and salts thereof: 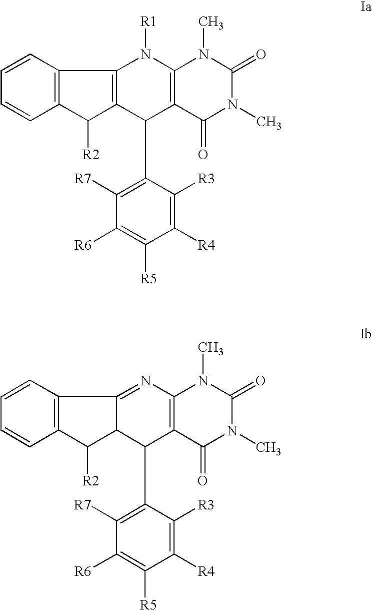
wherein R1 is —H or has the structure —C(═O)R8;
R2 is ═O or has the structure —OC(═O)R9; and
R3, R4, R5, R6, and R7 are each independently selected from the group consisting of —H, —NO₂, -halogen, —OC(═O)R9, —OR9, —OH, —R8OH, —CH₃, —OC(═O)CH₂Ph, 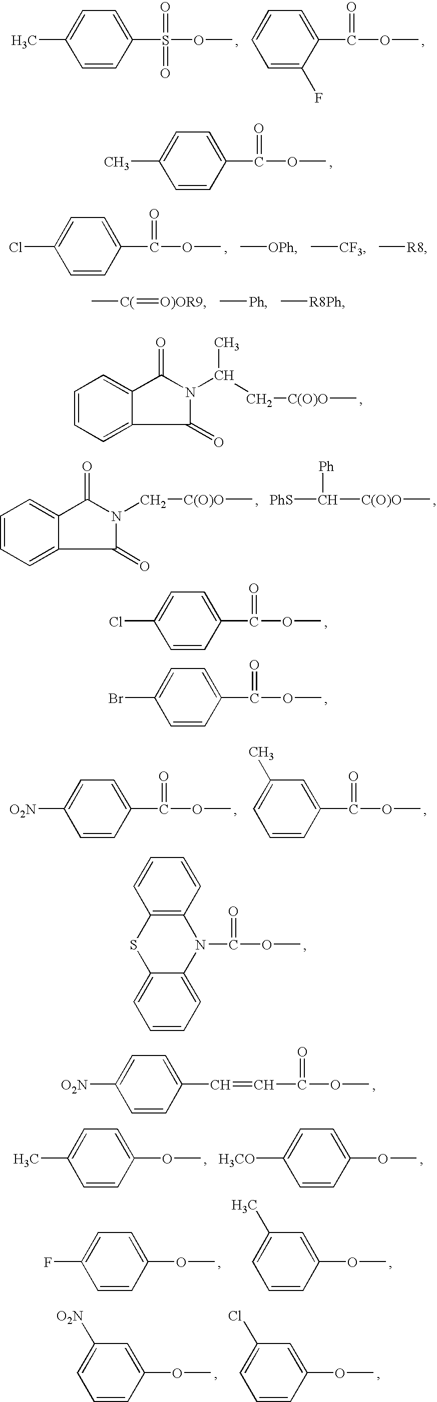 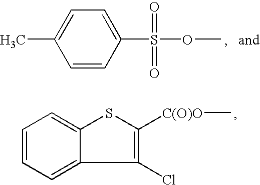
wherein each R8 is independently a linear or branched hydrocarbon group having from 1 to 4 carbon atoms and each R9 is independently a hydrocarbon group having from 1 to 2 carbon atoms.

2. The method of claim 1, wherein the at least one compound has structural formula Ia; R1 has the structure —C(═O)R8; R2 is ═O; R3 is —H; R4 is selected from the group consisting of —H, —NO₂, —Br, —OC(═O)CH₃, and —OR9; R5 is selected from the group consisting of —H, —NO₂, —F, —Cl, and —OC(═O)R9; R6 is —H or —OCH₃; and R7 is —H.

3. The method of claim 1, wherein the at least one compound has structural formula Ia; R1 is —H; R2 is ═O; R3 is —H; R4 is —Br; R5 is —H; R6 is —H; and R7 is —H.

4. The method of claim 1, wherein the at least one compound has structural formula Ib; R2 is —OC(═O)R9; R3 is —H; R4 is —H or —NO₂; R5 is selected from the group consisting of —H, —NO₂, —Br, —Cl, and —OCH₃; R6 is —H; and R7 is —H.

5. The method of claim 1, wherein the at least one compound has structural formula Ib; R2 is ═O; R3 is —H; R4 is —Br; R5 is —H; R6 is —H; and R7 is —H.

6. A method of treating a disease in a mammal mediated by activity of adenylyl cyclase or guanylyl cyclase and effected by a toxin produced by a pathogenic organism, comprising:
administering to the mammal an amount of a composition effective to treat the disease, wherein the composition comprises at least one compound selected from the group consisting of structural formulae Ia and Ib and salts thereof: 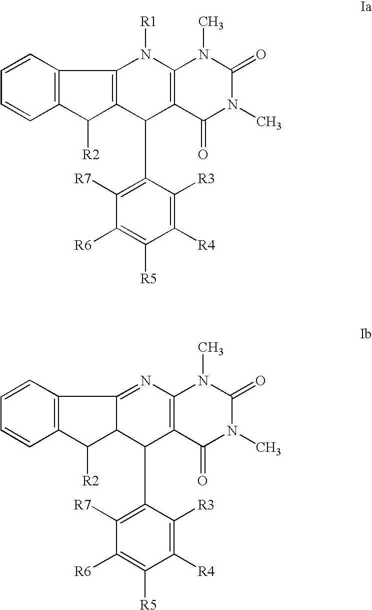
wherein R1 is —H or has the structure —C(═O)R8;
R2 is ═O or has the structure —OC(═O)R9; and
R3, R4, R5, R6, and R7 are each independently selected from the group consisting of —H, —NO₂, -halogen, —OC(═O)R9, —OR9, —OH, —R8OH, —CH₃, —OC(═O)CH₂Ph, 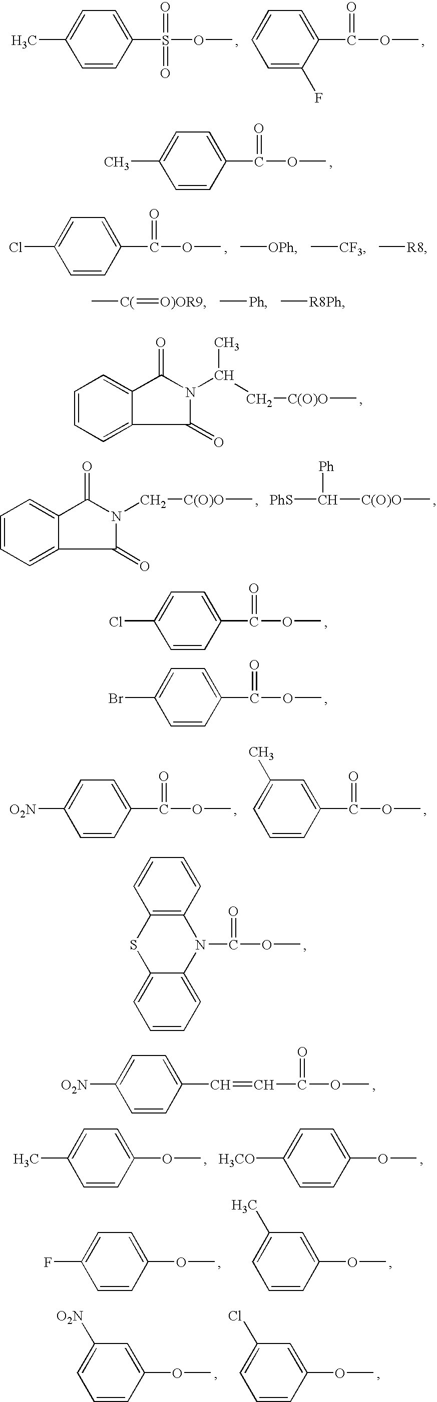 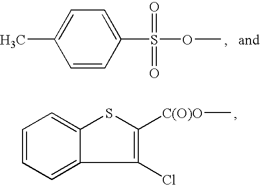
wherein each R8 is independently a linear or branched hydrocarbon group having from 1 to 4 carbon atoms and each R9 is independently a hydrocarbon group having from 1 to 2 carbon atoms.

7. The method of claim 6, wherein the at least one compound has structural formula Ia; R1 has the structure —C(═O)R8; R2 is ═O; R3 is —H; R4 is selected from the group consisting of —H, —NO₂, —Br, —OC(═O)CH₃, and —OR9; R5 is selected from the group consisting of —H, —NO₂, —F, —Cl, and —OC(═O)R9; R6 is —H or —OCH₃; and R7 is —H.

8. The method of claim 6, wherein the at least one compound has structural formula Ia; R1 is —H; R2 is ═O; R3 is —H; R4 is —Br; R5 is —H; R6 is —H; and R7 is —H.

9. The method of claim 6, wherein the at least one compound has structural formula Ib; R2 is —OC(═O)R9; R3 is —H; R4 is —H or —NO₂; R5 is selected from the group consisting of —H, —NO₂, —Br, —Cl, and —OCH₃; R6 is —H; and R7 is —H.

10. The method of claim 6, wherein the at least one compound has structural formula Ib; R2 is ═O; R3 is —H; R4 is —Br; R5 is —H; R6 is —H; and R7 is —H.

11. The method of claim 6, wherein the pathogenic organism is selected from the group consisting of Escherichia coli, Vibrio cholerae, Bordetella spp., Pseudomonas spp., Yersinia spp., and Bacillus anthracis.

12. The method of claim 6, wherein the disease is selected from the group consisting of diarrhea, cholera, pertussis, and anthrax.

13. The method of claim 12, wherein the disease is diarrhea and the mammal is selected from the group consisting of Bos spp., Sus spp., or Homo sapiens.

14. A method of reducing cyclic AMP or cyclic GMP levels in a mammal in need of reduction thereof, comprising:
administering to the mammal an amount of a composition effective to reduce the cyclic AMP or cyclic GMP level, wherein the composition comprises at least one compound selected from the group consisting of structural formulae Ia and Ib and salts thereof: 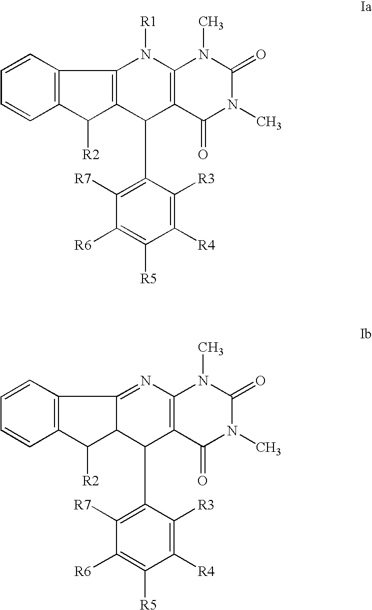
wherein R1 is —H or has the structure —C(═O)R8;
R2 is ═O or has the structure —OC(═O)R9; and
R3, R4, R5, R6, and R7 are each independently selected from the group consisting of —H, —NO₂, -halogen, —OC(═O)R9, —OR9, —OH, —R8OH, —CH₃, —OC(═O)CH₂Ph, 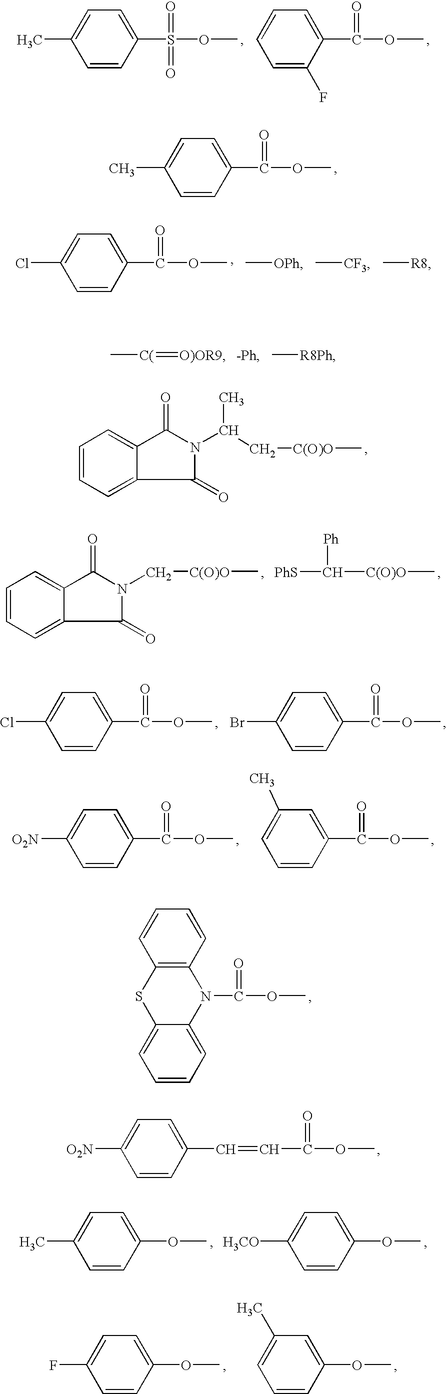 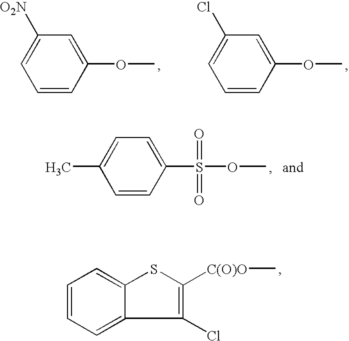
wherein each R8 is independently a linear or branched hydrocarbon group having from 1 to 4 carbon atoms and each R9 is independently a hydrocarbon group having from 1 to 2 carbon atoms.

15. The method of claim 14, wherein the at least one compound has structural formula Ia; R1 has the structure —C(═O)R8; R2 is ═O; R3 is —H; R4 is selected from the group consisting of —H, —NO₂, —Br, —OC(═O)CH₃, and —OR9; R5 is selected from the group consisting of —H, —NO₂, —F, —Cl, and —OC(═O)R9; R6 is —H or —OCH₃; and R7 is —H.

16. The method of claim 14, wherein the at least one compound has structural formula Ia; R1 is —H; R2 is ═O; R3 is —H; R4 is —Br; R5 is —H; R6 is —H; and R7 is —H.

17. The method of claim 14, wherein the at least one compound has structural formula Ib; R2 is —OC(═O)R9; R3 is —H; R4 is —H or —NO₂; R5 is selected from the group consisting of —H, —NO₂, —Br, —Cl, and —OCH₃; R6 is —H; and R7 is —H.

18. The method of claim 14, wherein the at least one compound has structural formula Ib; R2 is ═O; R3 is —H; R4 is —Br; R5 is —H; R6 is —H; and R7 is —H.

19. The method of claim 14, wherein the mammal suffers from a disease selected from the group consisting of cystic fibrosis, endocrinopathies, chronic obstructive pulmonary disease, adrenal cancer, pituitary cancer, lung cancer, chromaffin tumor, and parathyroid tumor.

20. A method of inhibiting activity of adenylyl cyclase or guanylyl cyclase in mammalian cells in vitro, comprising:
administering to the mammalian cells an amount of a composition effective to inhibit the activity, wherein the composition comprises at least one compound selected from the group consisting of structural formulae Ia and Ib and salts thereof: 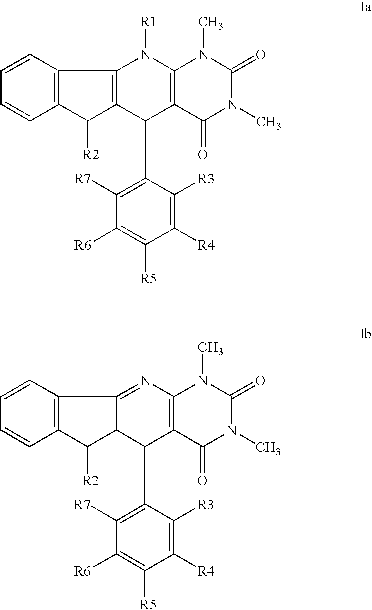
wherein R1 is —H or has the structure —C(═O)R8;
R2 is ═O or has the structure —OC(═O)R9; and
R3, R4, R5, R6, and R7 are each independently selected from the group consisting of —H, —NO₂, -halogen, —OC(═O)R9, —OR9, —OH, —R8OH, —CH₃, —OC(═O)CH₂Ph, 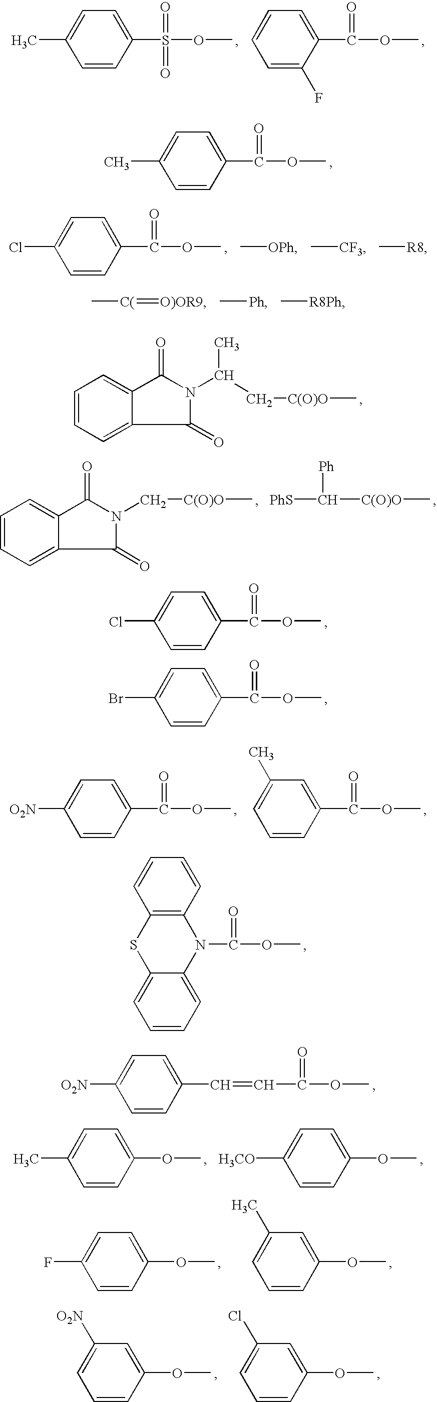 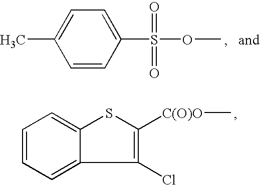
wherein each R8 is independently a linear or branched hydrocarbon group having from 1 to 4 carbon atoms and each R9 is independently a hydrocarbon group having from 1 to 2 carbon atoms.

21. The method of claim 20, wherein the at least one compound has structural formula Ia; R1 has the structure —C(═O)R8; R2 is ═O; R3 is —H; R4 is selected from the group consisting of —H, —NO₂, —Br, —OC(═O)CH₃, and —OR9; R5 is selected from the group consisting of —H, —NO₂, —F, —Cl, and —OC(═O)R9; R6 is —H or —OCH₃; and R7 is —H.

22. The method of claim 20, wherein the at least one compound has structural formula Ia; R1 is —H; R2 is ═O; R3 is —H; R4 is —Br; R5 is —H; R6 is —H; and R7 is —H.

23. The method of claim 20, wherein the at least one compound has structural formula Ib; R2 is —OC(═O)R9; R3 is —H; R4 is —H or —NO₂; R5 is selected from the group consisting of —H, —NO₂, —Br, —Cl, and —OCH₃; R6 is —H; and R7 is —H.

24. The method of claim 20, wherein the at least one compound has structural formula Ib; R2 is ═O; R3 is —H; R4 is —Br; R5 is —H; R6 is —H; and R7 is —H.
